(19) United States
(12) Patent Application Publication
(10) Pub. No.: US 2025/0127561 A1
(43) Pub. Date: Apr. 24, 2025

(54) ENERGY DELIVERY SYSTEMS AND USES THEREOF

(71) Applicant: NeuWave Medical, Inc., Madison, WI (US)

(72) Inventors: Mark Thom, Madison, WI (US); Matthew Thiel, Madison, WI (US)

(21) Appl. No.: 18/382,676

(22) Filed: Oct. 23, 2023

(51) Int. Cl. A61B 18/14; A61B 18/00; A61B 18/18

(52) U.S. Cl. CPC A61B 18/1492; A61B 18/1815; A61B 2018/00005; A61B 2018/00077; A61B 2018/00083; A61B 2018/00166; A61B 2018/00577; A61B 2018/00648; A61B 2018/00702; A61B 2018/00821; A61B 2018/1465; A61B 2018/1467; A61B 2018/1823; A61B 2560/02; A61B 2560/0468; A61B 2562/164

(57) ABSTRACT

Provided herein are devices configured for tissue ablation having hollow inner conductors for distal tip sensing access. In particular, energy delivery devices are provided having one or more sensors (e.g., positioning sensors, temperature sensors) positioned outside the distal end of a hollow inner conductor (e.g., within a region configured for ablation energy emission). In certain embodiments, such devices are utilized in systems and methods for delivering energy to tissue for a wide variety of applications, including medical procedures (e.g., tissue ablation, resection, cautery, vascular thrombosis, treatment of cardiac arrhythmias and dysrhythmias, electrosurgery, tissue harvest, etc.).

### FIELD OF THE INVENTION

Provided herein are devices configured for tissue ablation having hollow inner conductors for distal tip sensing access. In particular, energy delivery devices are provided having one or more sensors (e.g., positioning sensors, temperature sensors) positioned outside the distal end of a hollow inner conductor (e.g., within a region configured for ablation energy emission). In certain embodiments, such devices are utilized in systems and methods for delivering energy to tissue for a wide variety of applications, including medical procedures (e.g., tissue ablation, resection, cautery, vascular thrombosis, treatment of cardiac arrhythmias and dysrhythmias, electrosurgery, tissue harvest, etc.).

### BACKGROUND

In conventional microwave ablation probes, the distal radiating section of the antenna cannot be traversed by wire-based sensors due to the high e-field magnitude in the region causing electrical interference and/or unintended heating of the sensor wire. Existing microwave ablation probes on the market terminate sensors in a shielded region proximal to the antenna, which limits the physiological or ablation monitoring sensing that can be performed. The ability to place sensors at the leading distal tip of the probe and/or at the site of the energy delivery is potentially advantageous for several purposes: e.g. distal tip temperature sensing, tip-tracking/navigation aids, tissue dielectric property sensing, ablation growth monitoring.

The energy delivery devices described herein address these needs.

### SUMMARY

Energy delivery devices are provided having an energy delivery device proximal region, an energy delivery device most proximal end, an energy delivery device central region, an energy delivery device distal region, and an energy delivery device most distal end, wherein the energy delivery device does not proximally extend beyond the energy delivery device most proximal end, wherein the energy delivery device does not distally extend beyond the energy delivery device most distal end.

The energy delivery devices comprise:
a hollow inner conductor extending from the energy delivery device proximal region to the energy delivery device distal region, wherein the hollow inner conductor has a hollow inner conductor proximal region, a hollow inner conductor central region, and a hollow inner conductor distal region, wherein the hollow inner conductor proximal region has a hollow inner conductor most proximal end, wherein the hollow inner conductor distal region has a hollow inner conductor most distal end, wherein the hollow inner conductor does not extend proximally beyond the hollow inner conductor most proximal end, wherein the hollow inner conductor distal region does not distally extend beyond the hollow inner conductor most distal end; and
one or more sensors positioned within the energy delivery device distal region at or before the energy delivery device most distal end and beyond the hollow inner conductor most distal end, wherein the one or more sensors are selected from the group consisting of a temperature sensor, a positioning sensor, and an imaging sensor.

The energy delivery devices are configured to generate ablative energy (e.g., microwave energy) in a defined region surrounding the inner conductor distal region and the inner conductor most distal end, wherein generation of ablative energy does not compromise the function of the positioned one or more sensors.

Energy delivery devices having temperature sensors utilize temperature sensors designed to measure the temperature of the energy delivery device and/or tissue contacting the energy delivery device.

Energy delivery devices having positioning sensors utilize positioning sensors designed to measure the positioning of the energy delivery device.

Energy delivery devices having imaging sensors utilize imaging sensors designed to image the energy delivery device and/or tissue contacting the energy delivery device.

The energy delivery devices have a linear shape extending from the energy delivery device most proximal end to the energy delivery device most distal end.

The energy delivery devices may further comprise an outer conductor positioned along the exterior of the hollow inner conductor exterior; wherein the outer conductor extends from the energy delivery device most proximal end to the energy delivery device proximal region, or wherein the outer conductor extends from the energy delivery device most proximal end to the energy delivery device central region, or wherein the outer conductor extends from the energy delivery device most proximal end to the energy delivery device distal region, or wherein the outer conductor extends from the energy delivery device most proximal end to the energy delivery device most distal end.

The energy delivery devices may further comprise a dielectric portion positioned between the outer conductor and the hollow inner conductor.

Systems are provided comprising an energy delivery device and one or more of: a delivery tube, a microwave generator, a coolant supply, and a control computer.

Methods of ablating a tissue are provided comprising positioning an energy delivery device most distal end near a target tissue and applying ablative energy from the device while receiving information from the one or more sensors.

Additional embodiments are described herein.

### BRIEF DESCRIPTION OF THE DRAWINGS

FIG. 1 depicts an energy delivery device 1 having a hollow inner conductor 30 and positioned sensors 70.

FIG. 2 depicts a top-down-side view of an energy delivery device 1 showing a defined ablative emission region 60, a defined ablative energy emission protected region 65, and positioned sensors 70.

FIG. 3 depicts an energy delivery device 1 having a hollow inner conductor 30, an outer conductor 80, and positioned sensors 70.

FIG. 4 depicts an energy delivery device 1 having a hollow inner conductor 30, an outer conductor 80, a dielectric region 85, and positioned sensors 70.

FIG. 5 depicts an energy delivery device 1 having a hollow inner conductor 30, a dielectric region 85, and positioned sensors 70.

### DETAILED DESCRIPTION

Since high frequency currents only travel on the outer skin of conductors, the center of a cylindrical conductor is not necessary for microwave propagation. It is also an area of zero field, since no current flows in this region. Additionally, almost all microwave antennas feature an extension of the inner conductor wire to the distal tip of the probe. A hollow inner conductor feedthrough represents a novel and advantageous path to pass wires to the distal tip without passing through a region of high microwave field strength, allowing sensing/powered transducers at the distal tip of a microwave ablation probe.

Provided herein are devices configured for tissue ablation having hollow inner conductors for distal tip sensing access. In particular, energy delivery devices are provided having one or more sensors (e.g., positioning sensors, temperature sensors) positioned outside the distal end of a hollow inner conductor (e.g., within a region configured for ablation energy emission). Such energy delivery devices are utilized in systems and methods for delivering energy to tissue for a wide variety of applications, including medical procedures (e.g., tissue ablation, resection, cautery, vascular thrombosis, treatment of cardiac arrhythmias and dysrhythmias, electrosurgery, tissue harvest, etc.).

The energy delivery devices described herein (e.g., devices, antennae, transmission lines, etc.) solve several existing problems.

For example, the energy delivery devices described herein improve probe placement, and improve probe navigation systems. To enable probe needle navigation, it is most effective to have a sensor/emitter positioned in the distal tip of the probe. Needle tip tracking technology exists, but it is not implemented or integrated into microwave ablation needles due to incompatibility of the sensor/emitter with the electromagnetic (EM) field produced by a microwave antenna.

A distal temperature sensor allows temperature feedback during an ablation procedure to prevent damage to critical structures and/or provide feedback to know when necrotic temperatures have been reached. Current temperature sensors used are affected by the microwave antenna region and can't be placed most distal on the probe due to the EM field from the antenna.

Heat based ablations are affected by variable heat sinks in the body (proximity to blood vessels, tissue boundaries etc.). There is a clinical unmet need to measure physiological properties such as the dielectric constant of tissue, conductivity, elasticity etc., that could be used for better ablation outcome prediction and/or sensing.

The energy delivery devices described herein remedy such problems.

During medical procedures involving tissue ablation with ablation devices (e.g., microwave ablation devices) (e.g., radiofrequency ablation devices) ablation device placement (e.g., positioning) and the targeted ablation region are approximated using imaging and historical ablation data. A significant undesired side effect is the burning of tissue outside of the targeted ablation region. Indeed, when ablating near critical structures it can be difficult to encompass an intended target without damaging the critical structures. The energy delivery devices are capable of a) measuring a temperature at or near the device (e.g., a tissue region in contact with the device), and b) regulating the temperature (e.g., increasing, maintaining, or reducing) at or near the device. For example, such energy delivery devices can be used during an ablation procedure for purposes of monitoring tissue regions outside of the targeted ablation region and ensuring that such tissue regions do not experience tissue burning.

The energy delivery devices are not limited to particular size dimensions. Indeed, the size dimension of the energy delivery devices is such that it is able to fit within and pass through the lumen of a primary catheter (e.g., an endoscope). In addition, the size dimension of the energy delivery devices is such that it is able to be percutaneously inserted into a living mammal (e.g., living human being), and positioned at internal tissue region within the living mammal. The diameter of an energy delivery device is less than 5 mm (e.g., 5 mm or less, 4 mm or less, 3 mm or less, 2 mm or less, 1.4 mm or less, etc.). The energy delivery devices are of sufficient length to extend from an insertion site (e.g. mouth, incision into body of subject, etc.) to a desired target region within a living body (e.g. 50 cm . . . 75 cm . . . 1 m . . . 1.5 m . . . 2 m . . . 10 m . . . 25 m, etc.). The energy delivery devices are of sufficient length to extend through and beyond the reach of a primary catheter (e.g., endoscope) to reach a treatment site (e.g., peripheral lung tissue, heart tissue, gastrointestinal tissue, etc.) (e.g., any desired location within a living body).

The energy delivery devices are not limited to a particular manner of navigation through a primary catheter and/or through a body region. The energy delivery devices may utilize a navigation and/or steering mechanism, or may not utilize an independent means of navigation, position recognition, or maneuvering. The energy delivery devices may rely upon a primary catheter (e.g., endoscope) or a steerable navigation catheter for placement.

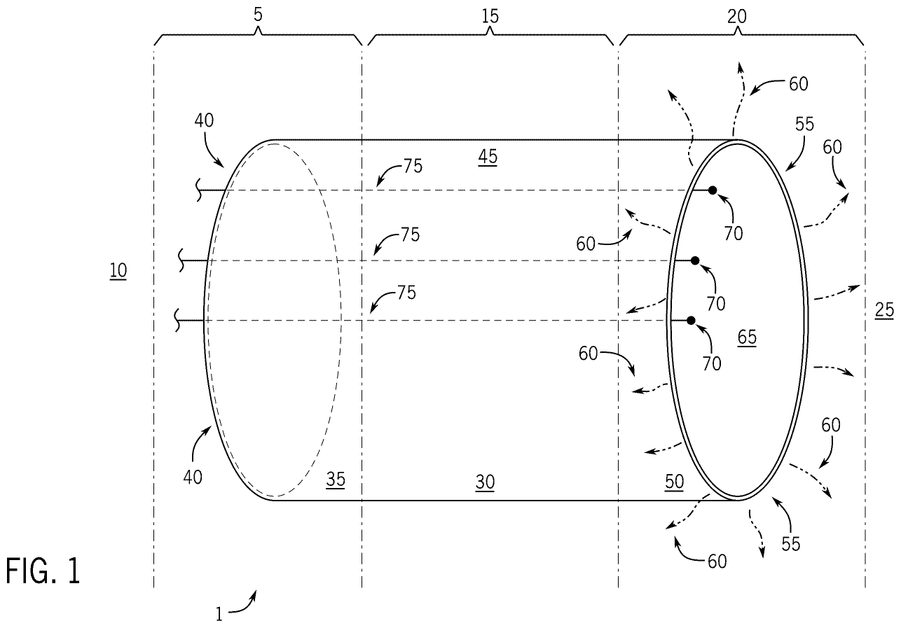

FIG. 1 depicts an energy delivery device 1. The energy delivery device 1 is not limited to a particular design or configuration. The design or configuration of the energy delivery device 1 is such that it is able to be positioned at a desired tissue region during medical procedures and emit ablation energy while operating one or more sensors (e.g., temperature sensor, position sensor, imaging sensor) positioned within the energy delivery device 1 (described in more detail below). The energy delivery device 1 has sufficient flexibility to access a circuitous route through a subject (e.g., through a branched structure, through a bronchial tree, through any region of the body to reach a desired location).

Still referring to FIG. 1, the energy delivery device 1 has an energy delivery device proximal region 5, an energy delivery device most proximal end 10, an energy delivery device central region 15, an energy delivery device distal region 20, and an energy delivery device most distal end 25. As shown, the energy delivery device 1 does not extend proximally beyond the energy delivery device most proximal end 10 (e.g., the energy delivery device most proximal end 10 is the start, initiation, origin of the energy delivery device 1). As shown, the energy delivery device most proximal end 10 is open. As shown, the energy delivery device 1 does not distally extend beyond the energy delivery device most distal end 25 (e.g., the energy delivery device most distal end 25 is the terminus of the energy delivery device 1).

The energy delivery device 1 is not limited to a particular amount of flexibility (e.g., can be bent or shaped in any direction or amount without compromising the function of the energy delivery device 1) or rigidity (e.g., cannot be bent or shaped in any manner without compromising the function of the energy delivery device 1) (e.g., the entire energy delivery device 1 is rigid) (e.g., the entire energy delivery device 1 is flexible) (e.g., portions of the energy delivery device 1 are rigid and portions of the energy delivery device 1 are flexible) (e.g., the energy delivery device proximal region 5 is flexible while the energy delivery device central region 15 and energy delivery device distal region 20 are each rigid) (e.g., the energy delivery device proximal region 5 and energy delivery device central region 15 are each flexible while the energy delivery device distal region 20 is rigid) (e.g., the energy delivery device proximal region 5 and the energy delivery device distal region 20 are each flexible while the energy delivery device central region 15 is rigid) (e.g., the energy delivery device proximal region 5 and energy delivery device distal region 20 are each rigid while the energy delivery device central region 15 is flexible) (e.g., the energy delivery device proximal region 5 and the energy delivery device central region 15 are each rigid while energy delivery device distal region 20 is flexible) (e.g., the energy delivery device proximal region 5 is rigid and the energy delivery device central region 15 and the energy delivery device distal region 20 are each flexible).

Still referring to FIG. 1, the energy delivery device 1 comprises a hollow inner conductor 30 having a hollow inner conductor proximal region 35, a hollow inner conductor most proximal end 40, a hollow inner conductor central region 45, a hollow inner conductor distal region 50, and a hollow inner conductor most distal end 55. As shown, the hollow inner conductor proximal region 35 is positioned within the energy delivery device proximal region 5. As shown, the hollow inner conductor most proximal end 40 is positioned at or near (e.g., within 0.001 mm, 0.01 mm, 0.1 mm, 0.2 mm, 0.5 mm, 1 mm, 1.5 mm, 5 mm, 10 mm) the energy delivery device most proximal end 10. As shown, the hollow inner conductor most proximal end 40 is open. As shown, the hollow inner conductor central region 45 is positioned within the energy delivery device central region 15. As shown, the hollow inner conductor distal region 50 is positioned within the energy delivery device distal region 20. As shown, the hollow inner conductor most distal end 55 is positioned at or near (e.g., within 0.1 mm, 0.2 mm, 0.5 mm, 1 mm, 1.5 mm, 5 mm, 10 mm) the energy delivery device most distal end 25. As shown, the hollow inner conductor most distal end 55 is open. As shown, the hollow inner conductor most proximal end 40 is the start, initiation, origin of the hollow inner conductor 30. As shown, the hollow inner conductor most distal end 55 is the terminus of the hollow inner conductor 30.

The design or configuration of the hollow inner conductor 30 is such that it is able to be positioned at a desired tissue region during medical procedures and emit ablation energy while operating one or more sensors (e.g., temperature sensor, position sensor, imaging sensor) positioned within the hollow inner conductor 30 (described in more detail below). As shown in FIG. 1, the hollow inner conductor 30 has a linear shape extending from the hollow inner conductor most proximal end 40 to the hollow inner conductor most distal end 55. The hollow inner conductor 30 is not limited to a particular amount of flexibility (e.g., can be bent or shaped in any direction or amount without compromising the function of the energy delivery device 1) or rigidity (e.g., cannot be bent or shaped in any manner) (e.g., the flexibility and/or rigidity of the hollow inner conductor 30 is consistent with the flexibility and/or rigidity of the energy delivery device 1) (e.g., the entire hollow inner conductor 30 is rigid) (e.g., the entire hollow inner conductor 30 is flexible) (e.g., portions of the hollow inner conductor 30 are rigid and portions of the hollow inner conductor 30 are flexible) (e.g., the hollow inner conductor proximal region 35 is flexible while the hollow inner conductor central region 45 and hollow inner conductor distal region 50 are each rigid) (e.g., the hollow inner conductor proximal region 35 and hollow inner conductor central region 45 are each flexible while the hollow inner conductor distal region 50 is rigid) (e.g., the hollow inner conductor proximal region 35 and the hollow inner conductor distal region 50 are each flexible while the hollow inner conductor central region 45 is rigid) (e.g., the hollow inner conductor proximal region 35 and the hollow inner conductor distal region 50 are each rigid while the hollow inner conductor central region 45 is flexible) (e.g., the hollow inner conductor proximal region 35 and the hollow inner conductor central region 45 are each rigid while the hollow inner conductor distal region 50 is flexible) (e.g., the hollow inner conductor proximal region 35 is rigid while the hollow inner conductor central region 45 and the hollow inner conductor distal region 50 are each flexible).

Still referring to FIG. 1, the energy delivery device 1 is configured to generate and emit ablative energy (e.g., microwave energy) (e.g., radiofrequency energy) in a defined ablative energy emission region 60 outside of and surrounding the hollow inner conductor distal region 50 and the hollow inner conductor most distal end 55, but not inside the hollow inner conductor distal region 50. As shown in FIG. 1, the defined ablative energy emission region 60 is shown with arrows positioned outside of and surrounding the hollow inner conductor distal region 50 and the hollow inner conductor most distal end 55, but not inside the hollow inner conductor distal region 50. Indeed, the defined ablative emission region 60 radiates outward (not inward) from the exterior of the hollow conductor distal region 50 and the hollow inner conductor most distal end 55 thereby creating a defined ablative energy emission protected region 65 interior to but not within the defined ablative energy emission region 60 that is not exposed to the defined ablative energy emission region 60. As shown, the defined ablative energy emission protected region 65 is beyond the hollow inner conductor most distal end 55, and within the energy delivery device distal region 20 at or before the energy delivery device most distal end 25.

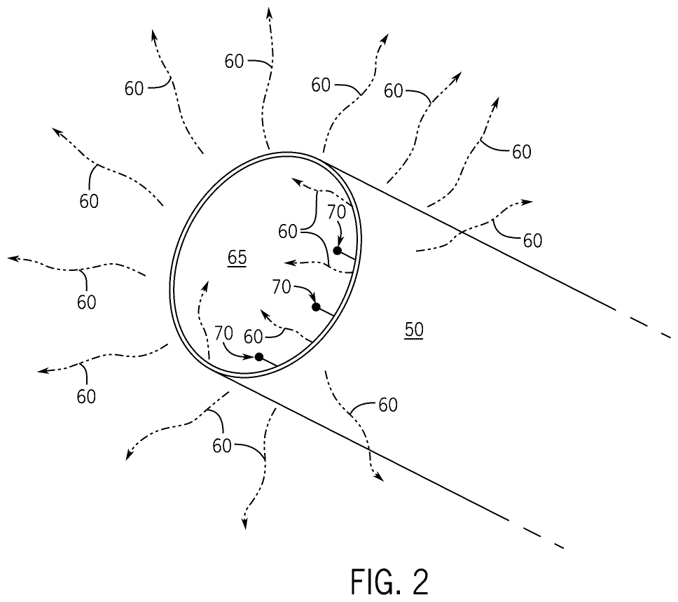

FIG. 2 depicts a top-down-side view of the defined ablative emission region 60 and sensors 70 positioned within the defined ablative energy emission protected region 65. As shown, arrows are utilized to show the defined ablative emission region 60 radiating outward (not inward) from the exterior of the hollow conductor distal region 50. As shown, the defined ablative energy emission protected region 65 is shown beyond the hollow inner conductor most distal end, and within the energy delivery device distal region at or before the energy delivery device most distal end. As shown, the defined ablative energy emission protected region 65 is shown interior to but not within the defined ablative energy emission region 60 and thereby not exposed to the defined ablative energy emission region 60. As shown, the sensors 70 are positioned within defined ablative energy emission protected region 65 but not within the defined ablative energy emission region 60 and thereby not exposed to the defined ablative energy emission region 60.

Referring again to FIG. 1, the energy delivery device 1 has therein one or more sensors 70 positioned 1) beyond the hollow inner conductor most distal end 55 and within the energy delivery device distal region 20 but not beyond the energy delivery device most distal end 25, and 2) within the defined ablative energy emission protected region 65. As shown in FIG. 1, each sensor 70 has a sensor thread line wire 75 extending from the sensor 70 and through the entirety of the inside of the hollow inner conductor 30 and outside of the hollow inner conductor most proximal end 40. Positioning of the each of the one or more sensors 70 involves threading the sensor 70 and respective sensor thread line wire 75 into the hollow inner conductor most proximal end 40, through the inside of the hollow inner conductor proximal region 35, through the inside of the hollow inner conductor central region 45, through the hollow inner conductor distal region 50, beyond the hollow inner conductor most distal end 55, and positioned 1) within the energy delivery device distal region 20 at or before the energy delivery device most distal end 25, and 2) within the defined ablative energy emission protected region 65. As shown in FIG. 1, the sensors 70 are positioned within the defined ablative energy emission protected region 65 such that emission of such ablative energy does not compromise the function of the sensors 70 (e.g., the sensors 70 are not exposed to the defined ablative energy emission region 60).

The energy delivery devices are not limited to a particular type of sensor or number of sensors. For example, an energy delivery device may have one sensor or a plurality of sensors (e.g., 2, 3, 4, 5, 6, 7, 8, 9, 10, 15, 20, 50, 100, etc). The sensor may record and/or report back any number of properties, including, but not limited to, heat at one or more positions of a component of the system, heat at the tissue, property of the tissue, and the like. The sensor may be in the form of an imaging device such as CT, ultrasound, magnetic resonance imaging, or any other imaging device. In some embodiments, particularly for research application, the system records and stores the information for use in future optimization of the system generally and/or for optimization of energy delivery under particular conditions (e.g., patient type, tissue type, size and shape of target region, location of target region, etc.).

The sensor may be a temperature sensor. The sensor may be a positioning sensor. The sensor may be an imaging sensor. An energy delivery device may have a temperature sensor and a positioning sensor. An energy delivery device may have a temperature sensor and an imaging sensor. An energy delivery device may have a positioning sensor and an imaging sensor. An energy delivery device may have a temperature sensor, a positioning sensor, and an imaging sensor. An energy delivery device may have only a temperature sensor. An energy delivery device may have only a positioning sensor. An energy delivery device may have only an imaging sensor.

An energy delivery device having a temperature sensor utilizes a temperature sensor designed to measure the temperature of the energy delivery device and/or tissue contacting the energy delivery device. As a temperature reaches a certain level the temperature sensor is designed to communicate a warning to a user via, for example, a processor.

Energy delivery device systems are provided that utilize temperature monitoring systems. Energy delivery devices are provided with temperature sensors positioned within the defined ablative energy emission protected region.

Temperature monitoring systems are used to accomplish one or more of the following: monitoring the temperature of an energy delivery device (e.g., with a temperature sensor), monitoring the temperature of a tissue region (e.g., tissue being treated, surrounding tissue), communicating with a processor for purposes of providing temperature information to a user or to the processor to allow the processor to adjust the system appropriately. The temperature sensors measure temperature with thermocouples or electromagnetic means through the energy delivery device. Data collected from temperature monitoring is used to initiate one or more cooling procedures described herein (e.g., coolant flow, lowered power, pulse program, shutoff, etc.).

An energy delivery device having a positioning sensor utilizes a positioning sensor designed to measure and indicate the precise location of the energy delivery device to a user via, for example, a processor.

An energy delivery device having an imaging sensor utilizes an imaging sensor designed to image (e.g., static images, real-time images) the energy delivery device and/or tissue contacting the energy delivery device, and indicate such imaging to a user via, for example, a processor.

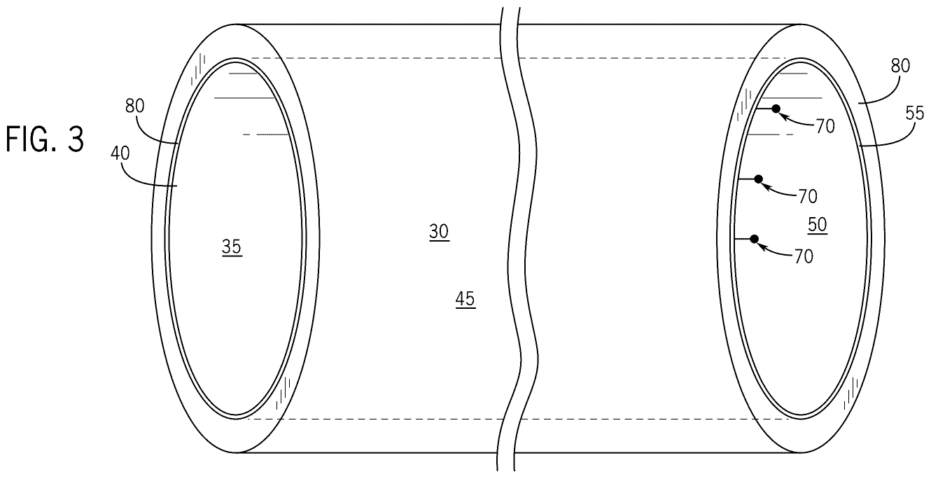

FIG. 3 depicts an energy delivery device 1 having an outer conductor 80 extending along the exterior of the hollow inner conductor 30 (e.g., along the exterior of the hollow inner conductor 30 from the hollow inner conductor most proximal end 40 and to the hollow inner conductor proximal region 35) (e.g., along the exterior of the hollow inner conductor 30 from the hollow inner conductor most proximal end 40 and to the hollow inner conductor central region 45) (e.g., along the exterior of the hollow inner conductor 30 from the hollow inner conductor most proximal end 40 and to the hollow inner conductor distal region 50). As shown in FIG. 3, the outer conductor 80 is positioned along the exterior of the hollow inner conductor 30 and enveloping the hollow inner conductor 30 along the exterior from the hollow inner conductor proximal most proximal end 40 and to the hollow inner conductor most distal end 55. As shown, sensors 70 are positioned beyond the hollow inner conductor distal region 50 and the hollow inner conductor most distal end 55.

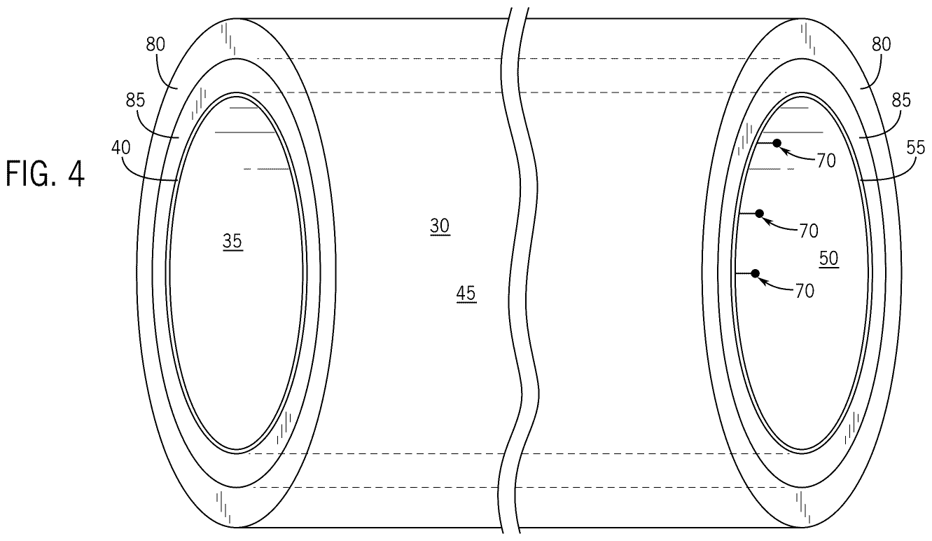

FIG. 4 depicts an energy delivery device 1 having dielectric region 85 positioned between an outer conductor 80 extending along the exterior of the hollow inner conductor 30 (e.g., along the exterior of the hollow inner conductor 30 from the hollow inner conductor most proximal end 40 and to the hollow inner conductor proximal region 35) (e.g., along the exterior of the hollow inner conductor 30 from the hollow inner conductor most proximal end 40 and to the hollow inner conductor central region 45) (e.g., along the exterior of the hollow inner conductor 30 from the hollow inner conductor most proximal end 40 and to the hollow inner conductor distal region 50). As shown in FIG. 4, the dielectric region 85 is positioned between the outer conductor 80 and the exterior of the hollow inner conductor 30 along the exterior from the hollow inner conductor proximal most proximal end 40 and to the hollow inner conductor most distal end 55. As shown, sensors 70 are positioned beyond the hollow inner conductor distal region 50 and the hollow inner conductor most distal end 55.

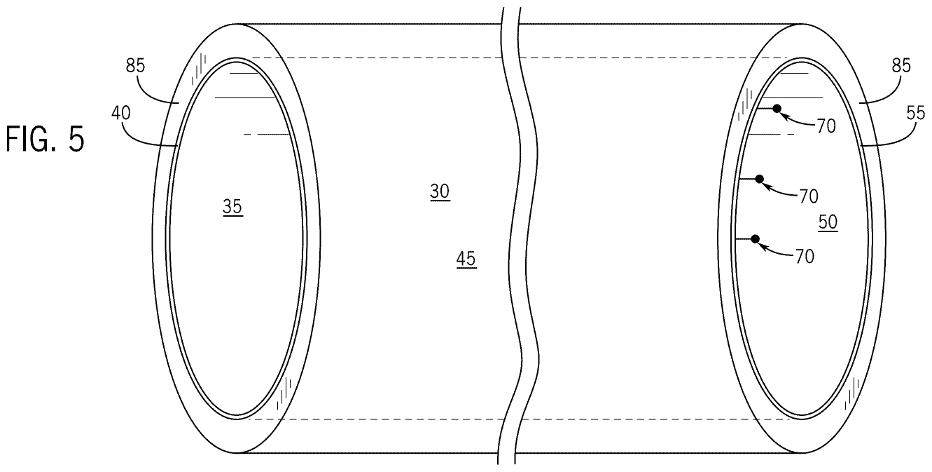

FIG. 5 depicts an energy delivery device 1 having dielectric region 85 extending along the exterior of the hollow inner conductor 30 (e.g., along the exterior of the hollow inner conductor 30 from the hollow inner conductor most proximal end 40 and to the hollow inner conductor proximal region 35) (e.g., along the exterior of the hollow inner conductor 30 from the hollow inner conductor most proximal end 40 and to the hollow inner conductor central region 45) (e.g., along the exterior of the hollow inner conductor 30 from the hollow inner conductor most proximal end 40 and to the hollow inner conductor distal region 50). As shown in FIG. 5, the dielectric region 85 is positioned along the exterior from the hollow inner conductor proximal most proximal end 40 and to the hollow inner conductor most distal end 55. As shown, sensors 70 are positioned beyond the hollow inner conductor distal region 50 and the hollow inner conductor most distal end 55.

Energy delivery devices are provided having variable lengths of an outer conductor and a dielectric. For example, energy delivery devices are provided where the dielectric extends further along the hollow inner conductor than the outer conductor. Energy delivery devices are provided where the outer conductor extends further along the hollow inner conductor than the dielectric. Energy delivery devices are provided having a dielectric and no outer conductor.

Energy delivery devices are provided wherein the positioning of the outer conductor, dielectric, and hollow inner conductor result in the energy delivery device having a coaxial transmission line configuration. The energy delivery devices are not limited to particular configurations of coaxial transmission lines. Examples of coaxial transmission lines include, but are not limited to, coaxial transmission lines developed by Pasternack, Micro-coax, and SRC Cables. Energy delivery devices and related systems are capable of utilizing antennae having flexible coaxial transmission lines (e.g., for purposes of positioning around, for example, pulmonary veins or through tubular structures) (see, e.g., U.S. Pat. Nos. 7,033,352, 6,893,436, 6,817,999, 6,251,128, 5,810,803, 5,800,494; each herein incorporated by reference in their entireties).

Energy delivery devices are provided wherein the outer conductor, dielectric, and hollow inner conductor result in the energy delivery device having a triaxial transmission line configuration. The energy delivery devices are not limited to particular configurations of triaxial transmission lines. A triaxial design has an outer conductor that allows improved tuning of the antenna to reduce reflected energy through the transmission line. This improved tuning reduces heating of the transmission line allowing more power to be applied to the tissue and/or a smaller transmission line (e.g., narrower) to be used. Further, the outer conductor may slide with respect to the hollow inner conductor to permit adjustment of the tuning to correct for effects of the tissue on the tuning. An energy delivery device with a triaxial design is sufficiently flexible to navigate a winding path (e.g. through a branched structure within a subject (e.g. through the brachial tree)).

Energy delivery devices are provided having a flexible and/or collapsible material (e.g. biaxially-oriented polyethylene terephthalate (boPET) (e.g. MYLAR, MELINEX, HOSTAPHAN, etc.), etc.).

Energy delivery devices are provided wherein the dielectric region material core is shaped to provide to provide channels within the dielectric space (e.g., air channels, coolant channels, vacant channels, etc.) having any desired design (e.g., channels are completely or partially encompassed by the dielectric material). Energy delivery devices are provided wherein the dielectric material divides the dielectric space into channels to create a “wagon wheel” conformation. Energy delivery devices are provided wherein the dielectric material divides the dielectric space (e.g. the space between the inner and outer conductors) into 1 or more channels (e.g. 1, 2, 3, 4, 5, 6, 7, 8, 9, 10, or more channels). Energy delivery devices are provided wherein the channels within the dielectric space serve as coolant channels (e.g., for circulating a coolant).

Energy delivery devices are provided wherein the channels within the dielectric space house coolant tubes. Energy delivery devices are provided wherein a first coolant channel delivers coolant along the length of the hollow inner conductor (e.g., from the hollow inner conductor most proximal end to the hollow inner conductor most distal end), and a second coolant channel provides the return path along the length of the hollow inner conductor (e.g., from the hollow inner conductor most distal end to the hollow inner conductor most proximal end). Energy delivery devices are provided wherein a channel comprises multiple coolant tubes (e.g., coolant and return).

Energy delivery devices are provided wherein a non-conductive jacket is positioned along the exterior (e.g., the entirety or only a portion) of the energy delivery devices. Energy delivery devices are provided wherein such a non-conductive jacket is positioned along the exterior (e.g., the entirety or only a portion) of the outer conductor. Energy delivery devices are provided wherein such a non-conductive jacket is positioned along the exterior (e.g., the entirety or only a portion) of the dielectric. Energy delivery devices are provided wherein such a non-conductive jacket is positioned along the exterior (e.g., the entirety or only a portion) of the hollow inner conductor.

Energy delivery devices are provided having a trocar positioned at the energy delivery device most distal end. Energy delivery devices are provided wherein the trocar is conductive. Energy delivery devices are provided wherein the trocar is not electrically connected to the hollow inner conductor. Energy delivery devices are provided wherein the trocar is capacitively coupled to the hollow inner conductor.

Energy delivery devices are provided configured to circulate a coolant (e.g., liquid) (e.g., water) (e.g., pressurized gas) (e.g., CO₂) (e.g., pressured CO₂) for purposes of regulating temperature (e.g., increasing, decreasing, maintaining) of the energy delivery device and/or a tissue region contacting the energy delivery device.

Energy delivery devices are provided wherein the energy delivery device is connectable (e.g., at the energy delivery device most proximal end) (e.g., at the energy delivery device proximal region) to an ablation energy generator (e.g., microwave ablation energy generator and/or a radiofrequency ablation energy generator), and/or a coolant source.

The use of microwave energy in the ablation of tissue has numerous advantages. For example, microwaves have a broad field of power density (e.g., approximately 2 cm surrounding an antenna depending on the wavelength of the applied energy) with a correspondingly large zone of active heating, thereby allowing uniform tissue ablation both within a targeted zone and in perivascular regions (see, e.g., International Publication No. WO 2006/004585; herein incorporated by reference in its entirety). In addition, microwave energy has the ability to ablate large or multiple zones of tissue using multiple probes with more rapid tissue heating. Microwave energy has an ability to penetrate tissue to create deep lesions with less surface heating. Energy delivery times are shorter than with radiofrequency energy and probes can heat tissue sufficiently to create an even and symmetrical lesion of predictable and controllable depth. Microwave energy is generally safe when used near vessels. Also, microwaves do not rely on electrical conduction as it radiates through tissue, fluid/blood, as well as air. Therefore, microwave energy can be used in tissue, lumens, lungs, and intravascularly.

Energy delivery devices are provided connectable a power supply that is an energy generator configured to provide as much as 100 watts of microwave power of a frequency of from 915 MHz to 5.8 GHZ. Energy delivery devices are provided connectable a a conventional magnetron of the type commonly used in microwave ovens is chosen as the generator. Energy delivery devices are provided wherein a single-magnetron based generator (e.g., with an ability to output 300 W through a single channel, or split into multiple channels) is utilized. It should be appreciated, however, that any other suitable microwave power source can substituted in its place. Energy delivery devices are provided connectable multiple types of generators including, but are not limited to, those available from Cober-Muegge, LLC, Norwalk, Connecticut, USA, Sairem generators, and Gerling Applied Engineering generators. Energy delivery devices are provided wherein the generator has at least approximately 60 Watts available (e.g., 50, 55, 56, 57, 58, 59, 60, 61, 62, 65, 70, 100, 500, 1000 Watts). For a higher-power operation, the generator is able to provide approximately 300 Watts (e.g., 200 Watts, 280, 290, 300, 310, 320, 350, 400, 750 Watts). Energy delivery devices are provided connectable with an energy generator able to provide as much energy as necessary (e.g., 400 Watts, 500, 750, 1000, 2000, 10,000 Watts). Energy delivery devices are provided connectable with an energy generator having solid state amplifier modules which can be operated separately and phase-controlled. Energy delivery devices are provided connectable with an energy generator having outputs combined constructively to increase total output power. Energy delivery devices are provided connectable with a power supply that distributes energy (e.g., collected from a generator) with a power distribution system. The energy delivery devices is not limited to a particular power distribution system. Energy delivery devices are provided connectable with a power distribution system that is configured to provide energy to an energy delivery device described herein for purposes of tissue ablation. The power distribution system is not limited to a particular manner of collecting energy from, for example, a generator. The power distribution system is not limited to a particular manner of providing energy to such energy delivery devices. Energy delivery devices are provided connectable with a power distribution system configured to transform the characteristic impedance of the generator such that it matches the characteristic impedance of an energy delivery device.

Energy delivery devices are provided connectable with a power distribution system configured with a variable power splitter so as to provide varying energy levels to different regions of an energy delivery device or to different energy delivery devices. Energy delivery devices are provided connectable with a power splitter provided as a separate component of the system. Energy delivery devices are provided connectable with a power splitter used to feed multiple energy delivery devices with separate energy signals. Energy delivery devices are provided connectable with a power splitter that electrically isolates the energy delivered to each energy delivery device so that, for example, if one of the devices experiences an increased load as a result of increased temperature deflection, the energy delivered to that unit is altered (e.g., reduced, stopped) while the energy delivered to alternate devices is unchanged.

Energy delivery devices are provided connectable with a power splitter designed by SM Electronics. Energy delivery devices are provided connectable with a power splitter configured to receive energy from a power generator and provide energy to additional system components (e.g., energy delivery devices). Energy delivery devices are provided connectable with a power splitter able to connect with one or more additional system components. Energy delivery devices are provided connectable with a power splitter configured to deliver variable amounts of energy to different regions within an energy delivery device for purposes of delivering variable amounts of energy from different regions of the device. Energy delivery devices are provided connectable with a power splitter used to provide variable amounts of energy to multiple energy delivery devices for purposes of treating a tissue region.

Energy delivery devices are provided to operate within a system comprising a processor, an energy delivery device, a temperature adjustment system, a power splitter (e.g., able to handle maximum generator outputs plus, for example, 25% (e.g., 20%, 30%, 50%)) (e.g., a 1000-watt-rated 2-4 channel power splitter), a tuning system, and/or an imaging system. Such power splitters are able to handle maximum generator outputs plus, for example, 25% (e.g., 20%, 30%, 50%).

Systems are provided wherein multiple energy delivery devices are employed, and the system may be configured to run them simultaneously or sequentially (e.g., with switching). Systems are provided configured to phase the fields for constructive or destructive interference. Phasing may also be applied to different elements within a single energy delivery device. Systems are provided wherein switching is combined with phasing such that multiple energy delivery devices are simultaneously active, phase controlled, and then switched to a new set of energy delivery devices (e.g., switching does not need to be fully sequential). Systems are provided wherein phase control is achieved precisely. Systems are provided wherein phase is adjusted continuously so as to move the areas of constructive or destructive interference in space and time. Systems are provided wherein the phase is adjusted randomly. Systems are provided wherein random phase adjustment is performed by mechanical and/or magnetic interference.

Energy delivery devices are provided having an anchoring element for securing the energy delivery device at a particular tissue region. Energy delivery devices are provided wherein the anchoring element is an inflatable balloon (e.g., wherein inflation of the balloon secures the antenna at a particular tissue region). An advantage of utilizing an inflatable balloon as an anchoring element is the inhibition of blood flow or air flow to a particular region upon inflation of the balloon. Such air or blood flow inhibition is particularly useful in, for example, cardiac ablation procedures and ablation procedures involving lung tissue, vascular tissue, and gastrointestinal tissue. Energy delivery devices are provided wherein the anchoring element is an extension of the energy delivery device designed to engage (e.g., latch onto) a particular tissue region. Further examples include, but are not limited to, the anchoring elements described in U.S. Pat. Nos. 6,364,876, and 5,741,249; each herein incorporated by reference in their entireties. Energy delivery devices are provided wherein the anchoring element has a circulating agent (e.g. a gas delivered at or near its critical point; CO₂) that freezes the interface between antenna and tissue thereby sticking the energy delivery device in place (e.g., as the tissue melts the energy delivery device remains secured to the tissue region due to tissue desiccation).

The energy delivery devices may be mounted onto additional medical procedure devices. For example, the energy delivery devices may be mounted onto endoscopes, intravascular catheters, bronchoscopes, or laparoscopes. The energy delivery devices may be mounted onto steerable catheters. The energy delivery devices may be deployed through endoscopes, intravascular catheters, bronchoscopes, or laparoscopes.

Energy delivery device systems are provided that utilize processors that monitor and/or control and/or provide feedback concerning one or more of the components of the system. Energy delivery systems are provided utilizing a processor provided within a computer module. The computer module may also comprise software that is used by the processor to carry out one or more of its functions. For example, the systems provide software for regulating the amount of microwave energy provided to a tissue region through monitoring one or more characteristics of the tissue region including, but not limited to, the size and shape of a target tissue, the temperature of the tissue region, and the like (e.g., through a feedback system) (see, e.g., U.S. Pat. Nos. 8,672,932, 10,363,092; and U.S. patent application Ser. No. 11/728,428; each of which is herein incorporated by reference in their entireties). Energy delivery systems are provided wherein the software is configured to provide information (e.g., monitoring information) in real time. Energy delivery systems are provided wherein the software is configured to interact with the energy delivery systems such that it is able to raise or lower (e.g., tune) the amount of energy delivered to a tissue region. Energy delivery systems are provided wherein the software is designed to prime coolants for distribution into, for example, an energy delivery device such that the coolant is at a desired temperature prior to use of the energy delivery device. Energy delivery systems are provided wherein the type of tissue being treated is inputted into the software for purposes of allowing the processor to regulate (e.g., tune) the delivery of microwave energy to the tissue region based upon pre-calibrated methods for that particular type of tissue region. Energy delivery systems are provided wherein the processor generates a chart or diagram based upon a particular type of tissue region displaying characteristics useful to a user of the system. Energy delivery systems are provided wherein the processor provides energy delivering algorithms for purposes of, for example, slowly ramping power to avoid tissue cracking due to rapid out-gassing created by high temperatures. Energy delivery systems are provided wherein the processor allows a user to choose power, duration of treatment, different treatment algorithms for different tissue types, simultaneous application of power to the antennas in multiple antenna mode, switched power delivery between antennas, coherent and incoherent phasing, etc. Energy delivery systems are provided wherein the processor is configured for the creation of a database of information (e.g., required energy levels, duration of treatment for a tissue region based on particular patient characteristics) pertaining to ablation treatments for a particular tissue region based upon previous treatments with similar or dissimilar patient characteristics. Energy delivery systems are provided wherein the processor is operated by remote control.

Energy delivery systems are provided wherein the processor is used to generate, for example, an ablation chart based upon entry of tissue characteristics (e.g., tumor type, tumor size, tumor location, surrounding vascular information, blood flow information, etc.) (e.g., the processor directs placement of the energy delivery device so as to achieve desired ablation based upon the ablation chart). Energy delivery systems are provided wherein a processor communicates with sensors (position sensors, temperature sensors, and/or imaging sensors) positioned within the defined ablative energy emission protected region and/or steering mechanisms to provide appropriate placement of systems and devices.

Energy delivery systems are provided wherein a software package (e.g., embodied in any desired form of non-transitory computer readable media) is provided to interact with the processor that allows the user to input parameters of the tissue to be treated (e.g., type of tumor or tissue section to be ablated, size, where it is located, location of vessels or vulnerable structures, and blood flow information) and then draw the desired ablation zone on a CT or other image to provide the desired results. The probes may be placed into the tissue, and the computer generates the expected ablation zone based on the information provided. Such an application may incorporate feedback. For example, CT, MRI, or ultrasound imaging or thermometry may be used during the ablation. This data is fed back into the computer, and the parameters readjusted to produce the desired result.

As used herein, the terms “computer memory” and “computer memory device” refer to any storage media readable by a computer processor. Examples of computer memory include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.).

As used herein, the term “computer readable medium” refers to any device or system for storing and providing information (e.g., data and instructions) to a computer processor. Examples of computer readable media include, but are not limited to, optical discs, magnetic disks, magnetic tape, solid-state media, and servers for streaming media over networks.

As used herein, the terms “processor” and “central processing unit” or “CPU” are used interchangeably and refer to a device that is able to read a program from a computer memory device (e.g., ROM or other computer memory) and perform a set of steps according to the program.

Energy delivery systems are provided that utilize imaging systems comprising imaging devices and/or software. The energy delivery systems are not limited to particular types of imaging devices (e.g., endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, electromagnetic imaging) (see, e.g., U.S. Pat. Nos. 6,817,976, 6,577,903, and 5,697,949, 5,603,697, and International Patent Application No. WO 06/005,579; each herein incorporated by reference in their entireties). Energy delivery systems are provided that utilize endoscopic cameras, imaging components, and/or navigation systems that permit or assist in placement, positioning, and/or monitoring of any of the items used with the energy systems described herein.

Energy delivery systems are provided with software configured for use of imaging equipment (e.g., CT, MRI, ultrasound). Energy delivery systems are provided wherein the imaging equipment software allows a user to make predictions based upon known thermodynamic and electrical properties of tissue, vasculature, and location of the antenna(s). Energy delivery systems are provided wherein the imaging software allows the generation of a three-dimensional map of the location of a tissue region (e.g., tumor, arrhythmia), location of the antenna(s), and to generate a predicted map of the ablation zone.

Energy delivery systems are provided wherein the imaging systems are used to monitor ablation procedures (e.g., microwave thermal ablation procedures, radio-frequency thermal ablation procedures). The energy delivery systems are not limited to a particular type of monitoring. Energy delivery systems are provided wherein the imaging systems are used to monitor the amount of ablation occurring within a particular tissue region(s) undergoing a thermal ablation procedure. Energy delivery systems are provided wherein the monitoring operates along with the ablation devices (e.g., energy delivery devices) such that the amount of energy delivered to a particular tissue region is dependent upon the imaging of the tissue region. The imaging systems are not limited to a particular type of monitoring. Energy delivery systems are provided wherein the monitoring is imaging blood perfusion for a particular region so as to detect changes in the region, for example, before, during and after a thermal ablation procedure. Energy delivery systems are provided wherein the monitoring includes, but is not limited to, MRI imaging, CT imaging, ultrasound imaging, nuclear medicine imaging, and fluoroscopy imaging. For example, prior to a thermal ablation procedure, a contrast agent (e.g., iodine or other suitable CT contrast agent; gadolinium chelate or other suitable MRI contrast agent, microbubbles or other suitable ultrasound contrast agent, etc.) is supplied to a subject (e.g., a patient) and the contrast agent perfusing through a particular tissue region that is undergoing the ablation procedure is monitored for blood perfusion changes. Energy delivery systems are provided wherein the monitoring is qualitative information about the ablation zone properties (e.g., the diameter, the length, the cross-sectional area, the volume). The imaging system is not limited to a particular technique for monitoring qualitative information. Energy delivery systems are provided wherein techniques used to monitor qualitative information include, but are not limited to, non-imaging techniques (e.g., time-domain reflectometry, time-of-flight pulse detection, frequency-modulated distance detection, eigenmode or resonance frequency detection or reflection and transmission at any frequency, based on one interstitial device alone or in cooperation with other interstitial devices or external devices). Energy delivery systems are provided wherein the interstitial device provides a signal and/or detection for imaging (e.g., electro-acoustic imaging, electromagnetic imaging, electrical impedance tomography). Energy delivery systems are provided wherein non-imaging techniques are used to monitor the dielectric properties of the medium surrounding the antenna, detect an interface between the ablated region and normal tissue through several means, including resonance frequency detection, reflectometry or distance-finding techniques, power reflection/transmission from interstitial antennas or external antennas, etc. Energy delivery systems are provided wherein the qualitative information is an estimate of ablation status, power delivery status, and/or simple go/no-go checks to ensure power is being applied. Energy delivery systems are provided wherein the imaging systems are designed to automatically monitor a particular tissue region at any desired frequency (e.g., per second intervals, per one-minute intervals, per ten-minute intervals, per hour-intervals, etc.). Energy delivery systems are provided with software designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the energy delivery devices. Likewise, an algorithm may be applied to predict the shape and size of the tissue region to be ablated (e.g., tumor shape) such that the system recommends the type, number, and location of ablation probes to effectively treat the region. Energy delivery systems are provided configured to operate with a navigation or guidance system (e.g., employing triangulation or other positioning routines) to assist in or direct the placement of the probes and their use.

For example, such procedures may use the enhancement or lack of enhancement of a contrast material bolus to track the progress of an ablation or other treatment procedure. Subtraction methods may also be used (e.g., similar to those used for digital subtraction angiography). For example, a first image may be taken at a first time point. Subsequent images subtract out some or all of the information from the first image so that changes in tissue are more readily observed. Likewise, accelerated imaging techniques may be used that apply “under sampling” techniques (in contrast to Nyquist sampling). It is contemplated that such techniques provide excellent signal-to-noise using multiple low resolutions images obtained over time. For example, an algorithm called HYPER (highly constrained projection reconstruction) is available for MRI that may be applied.

As thermal-based treatments coagulate blood vessels when tissue temperatures exceed, for example, 50° C., the coagulation decreases blood supply to the area that has been completely coagulated. Tissue regions that are coagulated do not enhance after the administration of contrast. Energy delivery systems are provided utilizing imaging systems to automatically track the progress of an ablation procedure by giving, for example, a small test injection of contrast to determine the contrast arrival time at the tissue region in question and to establish baseline enhancement. Energy delivery systems are provided wherein a series of small contrast injections is next performed following commencement of the ablation procedure (e.g., in the case of CT, a series of up to fifteen 10 ml boluses of 300 mg/ml water soluble contrast is injected), scans are performed at a desired appropriate post-injection time (e.g., as determined from the test injection), and the contrast enhancement of the targeted area is determined using, for example, a region-of-interest (ROI) to track any one of a number of parameters including, but not limited to, attenuation (Hounsfield Units [HU]) for CT, signal (MRI), echogenicity (ultrasound), etc. The imaged data is not limited to a particular manner of presentation. Energy delivery systems are provided wherein the imaging data is presented as color-coded or grey scale maps or overlays of the change in attenuation/signal/echogenicity, the difference between targeted and non-targeted tissue, differences in arrival time of the contrast bolus during treatment, changes in tissue perfusion, and any other tissue properties that can be measured before and after the injection of contrast material. Such methods are not limited to selected ROI's, but can be generalized to all pixels within any image. The pixels can be color-coded, or an overlay used to demonstrate where tissue changes have occurred and are occurring. The pixels can change colors (or other properties) as the tissue property changes, thus giving a near real-time display of the progress of the treatment. This method can also be generalized to 3d/4d methods of image display.

Energy delivery systems are provided wherein the area to be treated is presented on a computer overlay, and a second overlay in a different color or shading yields a near real-time display of the progress of the treatment. Energy delivery systems are provided wherein the presentation and imaging is automated so that there is a feedback loop to a treatment technology (RF, MW, HIFU, laser, cryo, etc) to modulate the power (or any other control parameter) based on the imaging findings. For example, if the perfusion to a targeted area is decreased to a target level, the power could be decreased or stopped. For example, such techniques are applicable to a multiple applicator system as the power/time/frequency/duty cycle, etc. is modulated for each individual applicator or element in a phased array system to create a precisely sculpted zone of tissue treatment. Conversely, the methods are used to select an area that is not to be treated (e.g., vulnerable structures that need to be avoided such as bile ducts, bowel, etc.). Energy delivery systems are provided wherein the methods monitor tissue changes in the area to be avoided, and warn the user (e.g., treating physician) using alarms (e.g., visible and/or audible alarms) that the structure to be preserved is in danger of damage. Energy delivery systems are provided wherein the feedback loop is used to modify power or any other parameter to avoid continued damage to a tissue region selected not to be treated. Energy delivery systems are provided wherein protection of a tissue region from ablation is accomplished by setting a threshold value such as a target ROI in a vulnerable area, or using a computer overlay to define a “no treatment” zone as desired by the user.

Energy delivery systems are provided that utilize tuning elements for adjusting the amount of energy delivered to the tissue region. Energy delivery systems are provided wherein the tuning element is manually adjusted by a user of the system. Energy delivery systems are provided wherein a tuning system is incorporated into an energy delivery device so as to permit a user to adjust the energy delivery of the device as desired (see, e.g., U.S. Pat. Nos. 5,957,969, 5,405,346; each herein incorporated by reference in their entireties). Energy delivery devices are provided pretuned to the desired tissue and is fixed throughout the procedure. Energy delivery systems are provided wherein the tuning system is designed to match impedance between a generator and an energy delivery device (see, e.g., U.S. Pat. No. 5,364,392; herein incorporated by reference in its entirety). Energy delivery systems are provided wherein the tuning element is automatically adjusted and controlled by a processor. Energy delivery systems are provided wherein a processor adjusts the energy delivery over time to provide constant energy throughout a procedure, taking into account any number of desired factors including, but not limited to, heat, nature and/or location of target tissue, size of lesion desired, length of treatment time, proximity to sensitive organ areas or blood vessels, and the like. Energy delivery systems are provided comprising a sensor that provides feedback to the user or to a processor that monitors the function of the device continuously or at time points.

Energy delivery devices may utilize a capacitor and/or energy gate at the hollow inner conductor distal region or hollow inner conductor most distal region. The capacitor and/or gate delivers energy (e.g. microwave energy) to the target site once a threshold of energy has built up behind the capacitor and/or gate. Low level energy is delivered along the transmission line, thereby reducing heat build-up along the pathway. Once sufficient energy has built up at the capacitor and/or gate, a high energy burst of energy (e.g. microwave energy) is delivered to the target site. The capacitor and/or gate delivery method has the advantage of reduced heating along the transmission path due to the low level energy transfer, as well as bursts of high energy being delivered at the target site (e.g. sufficient for tumor ablation).

All or a portion of the energy generating circuitry is located at one or more points along the hollow inner conductor. All or a portion of the microwave generating circuitry is located at one or more points along the hollow inner conductor. Generating energy (e.g. microwave energy) at one or more points along the hollow inner conductor reduces the distance the energy needs to travel, thereby reducing energy loss, and undesired heat generation. Generating energy (e.g. microwave energy) at one or more points along the transmission line allows for operating at reduced energy levels while providing the same energy level to the treatment site.

The energy delivery device systems are not limited to particular uses. Indeed, the energy delivery systems are designed for use in any setting wherein the emission of energy is applicable. Such uses include any and all medical, veterinary, and research applications. In addition, the systems and devices may be used in agricultural settings, manufacturing settings, mechanical settings, or any other application where energy is to be delivered.

Energy delivery devices are provided capable of accessing access to a difficult to reach region of the body (e.g., the periphery or central regions of the lungs). Energy delivery devices are provided capable of navigating through a branched body structure (e.g., bronchial tree) to reach a target site. Accessing the lungs through the bronchi provides a precise and accurate approach while minimizing collateral damage to the lungs. Accessing the lung (e.g. central lung or lung periphery) from outside the lung requires puncturing or cutting the lung, which can be avoided by bronchial access.

Energy delivery device systems are provided with a primary catheter (e.g. endoscope, bronchoscope, etc.), containing a channel catheter and steerable navigation catheter is advanced into the bronchial tree (e.g. via the trachea) until the decreasing circumference of the bronchi will not allow further advancement of the primary catheter. Energy delivery device systems are provided with a primary catheter (e.g. endoscope, bronchoscope, etc.), containing a channel catheter and steerable navigation catheter is advanced into the bronchial tree (e.g. via the trachea) up to the desired point for deployment of the channel catheter Energy delivery device systems are provided wherein the primary catheter is advanced into the trachea, primary bronchi, and/or secondary bronchi, but not further. Energy delivery device systems are provided with a channel catheter containing a steerable navigation catheter is advanced through the primary catheter, and beyond the distal tip of the primary catheter, into the bronchial tree (e.g. via the trachea, via the primary bronchi, via secondary bronchi, via tertiary bronchi, via bronchioles, etc.) up to the target location (e.g. treatment site, tumor, etc.). Energy delivery device systems are provided wherein a channel catheter containing a steerable navigation catheter is advanced into the bronchial tree (e.g. via the trachea, primary bronchi, etc.) until the decreasing size of the bronchi will not allow further advancement (e.g. in the tertiary bronchi, in the bronchioles, at the treatment site). Energy delivery device systems are provided wherein a channel catheter is advanced into the trachea, primary bronchi, secondary bronchi, tertiary bronchi, and/or bronchioles. Energy delivery device systems are provided wherein a steerable navigation catheter is advanced into the trachea, primary bronchi, secondary bronchi, tertiary bronchi, and/or bronchioles to the treatment site Energy delivery device systems are provided wherein a steerable navigation catheter is withdrawn through the channel catheter, leaving the open channel lumen extending from the point of insertion (e.g. into the subject, into the trachea, into the bronchial tree, etc.), through the bronchial tree (e.g. through the trachea, primary bronchi, secondary bronchi, tertiary bronchi, bronchioles, etc.) to the target site (e.g. treatment site, tumor, central or peripheral lunch tumor). Energy delivery device systems are provided wherein an energy delivery device (e.g. microwave ablation device) is inserted through the open channel lumen to access the target site.

Energy delivery device systems are provided wherein transbronchial treatment is employed. Energy delivery device systems are provided wherein the energy delivery device are positioned through the airways (e.g., following bronchial tree) to the best straight line or other desired path to the target. The airway wall is then pierced and the device is advanced in proximity to the target to facilitate ablation.

Energy delivery device systems are provided for placement of an energy delivery device at a difficult to access tissue region within a subject. Energy delivery device systems are configured to provide access to, and/or treatment of, tumors, growths, and/or nodules on the periphery of the lungs or in the central lungs. Energy delivery device systems are configured to provide access to, and ablation of, peripheral pulmonary nodules. Peripheral pulmonary nodules and central nodules are difficult to access through the bronchial tree because of their location near the tertiary bronchi and bronchioles, beyond the reach of conventional devices and techniques. Energy delivery device systems are configured to provide access to central and peripheral pulmonary nodules through the bronchial tree. Peripheral pulmonary nodules are generally less than 25 mm in diameter (e.g. <25 mm, <20 mm, <10 mm, <5 mm, <2 mm, <1 mm, etc.). Peripheral pulmonary nodules are 0.1 mm-25 mm in diameter (e.g. 0.1 mm . . . 0.2 mm . . . 0.5 mm . . . 1.0 mm . . . 1.4 mm . . . 2.0 mm . . . 5.0 mm . . . 10 mm . . . 20 mm . . . 25 mm, and diameters therein). . . . Energy delivery device systems are configured to provide access and treatment of tumors, growths, and nodules of any size and any location within a subject (e.g. within the lungs of a subject). Energy delivery device systems are configured to provide curative treatment and/or palliative treatment of tumors (e.g. nodules) in the central or peripheral lung.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. An energy delivery device having an energy delivery device proximal region, an energy delivery device most proximal end, an energy delivery device central region, an energy delivery device distal region, and an energy delivery device most distal end, wherein the energy delivery device does not proximally extend beyond the energy delivery device most proximal end, wherein the energy delivery device does not distally extend beyond the energy delivery device most distal end;
wherein the energy delivery device comprises:
a hollow inner conductor extending from the energy delivery device proximal region to the energy delivery device distal region, wherein the hollow inner conductor has a hollow inner conductor proximal region, a hollow inner conductor central region, and a hollow inner conductor distal region, wherein the hollow inner conductor proximal region has a hollow inner conductor most proximal end, wherein the hollow inner conductor distal region has a hollow inner conductor most distal end, wherein the hollow inner conductor does not extend proximally beyond the hollow inner conductor most proximal end, wherein the hollow inner conductor distal region does not distally extend beyond the hollow inner conductor most distal end;
one or more sensors positioned within the energy delivery device distal region at or before the energy delivery device most distal end and beyond the hollow inner conductor most distal end, wherein the one or more sensors are selected from the group consisting of a temperature sensor, a positioning sensor, and an imaging sensor;
wherein the energy delivery device is configured to generate ablative energy in a defined region surrounding the inner conductor distal region and the inner conductor most distal end.

2. The energy delivery device of claim 1, wherein the ablative energy is microwave energy and/or radiofrequency energy.

3. The energy delivery device of claim 1, wherein one or more of the one or more sensors positioned within the energy delivery device distal region at or before the energy delivery device most distal end and beyond the hollow inner conductor most distal end are capable of proper function during generation of ablation energy.

4. The energy delivery device of claim 1, wherein the temperature sensor is designed to measure the temperature of the energy delivery device and/or tissue contacting the energy delivery device.

5. The energy delivery device of claim 1, wherein the positioning sensor is designed to measure the positioning of the energy delivery device.

6. The energy delivery device of claim 1, wherein the imaging sensor is designed to image the energy delivery device and/or tissue contacting the energy delivery device.

7. The energy delivery device of claim 1, wherein the energy delivery device has a linear shape extending from the energy delivery device most proximal end to the energy delivery device most distal end.

8. The energy delivery device of claim 1, further comprising an outer conductor positioned along the exterior of the hollow inner conductor exterior;
wherein the outer conductor extends from the energy delivery device most proximal end to the energy delivery device proximal region, or
wherein the outer conductor extends from the energy delivery device most proximal end to the energy delivery device central region, or
wherein the outer conductor extends from the energy delivery device most proximal end to the energy delivery device distal region, or
wherein the outer conductor extends from the energy delivery device most proximal end to the energy delivery device most distal end.

9. The energy delivery device of claim 8, further comprising a dielectric portion positioned between the outer conductor and the hollow inner conductor.

10. The energy delivery device of claim 1, wherein at least a portion of the energy delivery device is flexible.

11. A system comprising an energy delivery device of claim 1 and one or more of: a delivery tube, a microwave generator, a coolant supply, and a control computer.

12. A method of ablating a tissue comprising: positioning the energy delivery device's most distal end of claim 1 near a target tissue and applying ablative energy from the device.
